(19) United States
(12) Patent Application Publication
(10) Pub. No.: US 2009/0074821 A1
(43) Pub. Date: Mar. 19, 2009

(54) OIL ABSORBING COSMETIC SHEET

(76) Inventors: Hiroshi Sakurai, Tokyo (JP); Yoshiyuki Matsumura, Tokyo (JP); Yukari Sekiya, Tokyo (JP)

Correspondence Address: 3M INNOVATIVE PROPERTIES COMPANY, PO BOX 33427, ST. PAUL, MN, 55133-3427, US

(21) Appl. No.: 11/813,158

(22) PCT Filed: Dec. 21, 2005
(86) PCT No.: PCT/US2005/046980

§ 371 Date: May 14, 2008

(30) Foreign Application Priority Data
Jan. 7, 2005 (JP) 2005-002552

(51) Int. Cl. A61K 8/02; A61Q 19/00

(52) U.S. Cl. 424/401

(57) ABSTRACT

To provide an oil-absorbing cosmetic sheet having very excellent oil absorption property and remarkably soft touch and enabling oil removal with enjoyment before and during use by virtue of an imparted picture pattern. The oil-absorbing cosmetic sheet comprises a porous stretched film formed of a plastic material and is constructed such that an image-forming oil agent, in an amount imparting a picture pattern to the plastic film by at least partially filling voids of the plastic film, is applied to the plastic film and thereby an image region is formed.

The present invention relates to an oil-absorbing cosmetic sheet, more specifically, the present invention relates to an oil-absorbing cosmetic sheet for wiping oil (sebum) oozed out of the face.

### BACKGROUND ART

Oil-absorbing papers of various types have been conventionally used for wiping sebum oozed out of the face, particularly the nose, cheek, forehead and middle forehead, to keep clean the face and allowing good applicability and spreadability of cosmetics. Conventional oil-absorbing papers on the market are papers formed using vegetable fibers such as hemp or synthetic pulp. Such oil-absorbing papers have high oil absorption, but are also highly irritating to the skin due to the hardness and surface roughness of the fibers. In order to decrease skin irritation during production the paper is calendered or coated with an inorganic powder such as calcium carbonate powder together with a sizing agent. However, calendering has the problem that the fibers raise with aging and can again irritate the skin. Coating has the problem that since the paper surface is covered by a sizing agent the sebum absorbing capacity decreases. These problems likewise occur in commercially available oil-absorbing paper comprising non-woven fabrics of thermoplastic resins.

In order to solve these problems, JPH-239517A invented an oil-absorbing cosmetic sheet, which comprises a porous stretched film of a thermoplastic material, the porous stretched film contains a filler, the interstitial volume per unit area of the porous stretched film is from 0.0001 to 0.005 cm³, and the pore diameter of voids occupying in the porous stretched film is from 0.2 to 5 μm. The oil-absorbing sheet was designed to allow for keeping the raw material filler in the film without removing it in the production process, excellent oil absorption no rupture during use, the visualization of the oil absorption effect by transparentization of the film with oil absorption, and soft touch. Powder particle coatings or the like are not needed. This oil-absorbing sheet has enjoyed rapid widespread popularity in recent years.

The same development group also invented an oil-absorbing wipe suitable for cleaning the skin or hair of a user as disclosed in WO 03/043590. This oil-absorbing wipe is characterized by comprising an oil-absorbing porous film-like substrate of a thermoplastic material, where the porous substrate changes in the transparency or color with oil absorption, and at least a part, at least 1%, of the surface area has an oil-base coating. With this oil-absorbing wipe, for example, a vegetable oil, a synthetic oil or a blend thereof is coated on the porous substrate surface, increasing the rapidness of film transparentization with small amounts of oil absorption as compared with films having no oil coating (a rapid oil absorption indicating function).

JP2003-204824A discloses an oil-absorbing cosmetic paper in which a transparentizing agent such as petroleum-derived hydrocarbon resin is coated in a printing pattern on a part of at least one surface of the paper. The paper has a bulk density of 0.6 or more and is obtained by blending (B) 50 parts by weight or less of an inorganic filler to (A) 100 parts by weight of a pulp raw material mainly comprising a vegetable fiber to prepare a paper stock and then paper. This Patent also discloses an oil-absorbing cosmetic paper in which a transparentizing agent such as petroleum-derived hydrocarbon resin and an oil-resistant agent such as fluorocarbon-based compound are coated in different printing patterns. In these oil-absorbing papers, the transparentizing agent is coated in a printing pattern and the printing pattern is transparentized and disappears with use, so that the oil absorption effect can be more clearly confirmed (indicator function).

### DISCLOSURE OF THE INVENTION

The oil-absorbing sheet of the present invention is excellent in the oil absorption, can easily indicate oil-absorption by the sheet turning more transparent, is soft to the touch, is less irritating to skin than paper and effectively cleans the face ensuring good applicability and spreadability of cosmetics. In particular, the oil-absorbing sheet of the present invention changes in transparency or color with a picture pattern imparted to the sheet. Before oil absorption, the user can see the picture pattern. After and during use the user can see the oil absorption and the change in the picture pattern. The portion imparted with the picture pattern has an oil-absorbing performance different from other portions. Allowing even a user having a relatively small amount of sebum on the face to see the oil absorption similarly to a user having a large amount of sebum.

With the widespread use of oil-absorbing papers, and sheet and the like (hereinafter referred to as “oil-absorbing sheets”) for cosmetics, oil-absorbing sheets having new additive values are demanded. As a result of studies to meet this demand, the present inventors perceived a desire to impart picture patterns on oil-absorbing sheets to increase users enjoyment. However, conventional oil-absorbing thermoplastic sheets are generally unsuitable for imparting picture patterns. For example, the oil-absorbing cosmetic sheet described in JP 11-239517A generally contains mineral oil filler which creates problems with fixing normal printing inks, which readily separated or do not allow picture patterns to be printed. Furthermore, even if an ink has good affinity for the oil-absorbing sheet, it is necessary to confirm whether or not the ink is safe for use with skin. Thus, printing with inks is not practical.

JP Patents 2003-204824A and 204825 proposed coating transparentizing agent in a printing pattern on an oil-absorbing paper formed of vegetable fibers and therefore, there is the problem of skin irritation due to hardness and surface roughness of the raw material fiber, which does not conform with the object of the present invention. Furthermore, whether or not the transparentizing agent used in these patent publications can provide a desired picture pattern with a porous stretched plastic film intended to use in the implementation of the present invention was not known.

Accordingly, an object of the present invention is to provide an oil-absorbing cosmetic sheet which is not only less irritating to the skin and excellent for cleaning the face but also has the added value of a picture pattern can be formed that can be enjoyed before and after use.

Another object of the present invention is to provide an oil-absorbing cosmetic sheet which can give an oil absorption indicating effect high enough to functionally and visually satisfy both users having a large amount of facial sebum and users having a relatively small amount of facial sebum.

As a result of intensive investigation to attain the above-described objects, the present inventors have found that it is very effective to form a oil-absorbing cosmetic sheet from an oil-absorbing porous stretched plastic film having voids formed with a filler and form a picture pattern by filling some of the voids with an oil agent having specific physical properties. The present invention has been accomplished based on this finding.

Accordingly, the present invention is an oil-absorbing cosmetic sheet comprising a porous stretched film formed of a plastic material, wherein an image-forming oil agent is applied to impart a picture pattern to the plastic film by at least partially filling voids of the plastic film to form an image region.

### BRIEF DESCRIPTION OF THE DRAWINGS

FIG. 1A perspective view showing one preferred embodiment of the oil-absorbing cosmetic sheet according to the present invention.

FIG. 2 A perspective view showing the state after the oil-absorbing cosmetic sheet of FIG. 1 is used.

### DESCRIPTION OF NUMERICAL REFERENCES

1 Porous stretched plastic film
10 Oil-absorbing cosmetic sheet
11 Image region
12 Image region
13 Image region

### EFFECTS OF THE INVENTION

As will be understood from the detailed description below the present invention provides an oil-absorbing cosmetic sheet which has excellent oil absorption, easily indicates oil-absorption by transparentization of the sheet, is soft to the touch, is not irritating to skin and has a high face cleaning effect.

Also, according to the present invention, the transparency or color of a picture pattern imparted to the sheet changes during oil absorption, so that the user can enjoy the presence of the picture pattern before use and the change in the picture pattern during use. Furthermore, an oil agent is already filled in the voids of the film, so that the oil absorption amount in the portion imparted with the picture pattern can be made smaller than that in the portion not having a picture pattern allowing even a user having a relatively small amount of sebum on the face can be fully satisfied that oil is removed similar to a user having a large amount of sebum.

The oil agent is safe to skin as compared with printing inks or the like, and are oils generally used, for example, in cosmetics which can be implemented easily and at a low cost.

In addition, the oil agent moves into voids without remaining on the surface of the oil-absorbing sheet and is held there, as a result, the surface is not tacky due to formation of a picture pattern and does not impair feel during use or cause blocking of the sheets.

Furthermore, the oil agent can be printed by a simple printing method such as flexographic printing or gravure printing to impart a picture pattern, so that the oil-absorbing cosmetic sheet can be simply and advantageously mass-produced.

### BEST MODE FOR CARRYING OUT THE INVENTION

The oil-absorbing cosmetic sheet of the present invention can be advantageously used in various modes. The present invention is described below by referring to typical embodiments thereof.

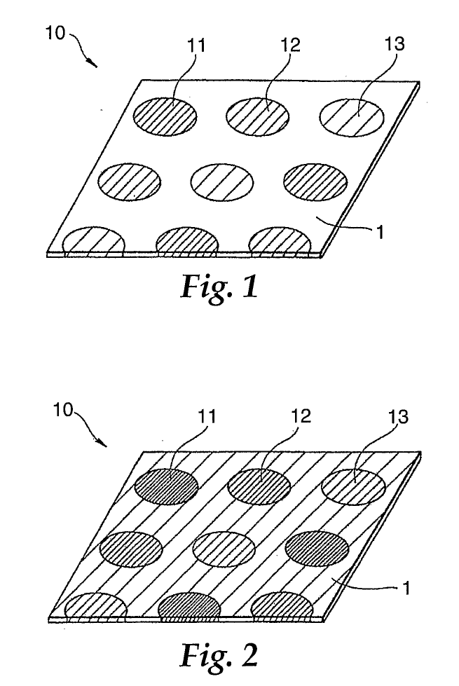

The oil-absorbing cosmetic sheet of the present invention is first roughly described by referring to FIGS. 1 and 2. As shown in FIG. 1, the oil-absorbing cosmetic sheet 10 of the present invention comprises a porous stretched plastic film substrate 1. In the inside of the plastic film 1, a large number of fine voids (in the present invention, sometimes referred to as “pores”) are almost uniformly dispersed, although are not shown. The voids are formed during production of the film.

The plastic film 1 is preferably colored with an arbitrary color serving the needs of users. Particularly, in the case of the oil-absorbing sheet 10 of the present invention, the plastic film 1 not only has an arbitrary color but also has picture patterns 11 to 13 each giving different visions before and after use. The picture pattern (in the present invention, this portion is particularly referred to as an “image region”) is formed by applying a specific image-forming oil agent to a predetermined image formation region (that is, a region to be imparted with a picture pattern). The oil agent can be applied by an any method but is preferably applied by coating, more preferably by printing. When the oil agent is applied, the oil agent fills in a part or all of the voids already formed in the plastic film 1 and a desired picture pattern is formed in the image formation region, which can be visually recognized. The oil-absorbing sheet 10 of the present invention comprises multiple species and multiple pieces (in the Figure, 3 species and 6 pieces) of picture patterns 11 to 13 differing in color, tint or color density as shown in the Figures or differing in pattern (not shown) and therefore, has a visually appealing appearance. As a result, the commercial value can be elevated and the user can enjoy the printed picture pattern, which cannot be experienced with conventional products.

FIG. 2 shows the oil-absorbing sheet 10 shown in FIG. 1 after use. In this Figure, to facilitate understanding of the oil absorption activity, sebum is shown as almost uniformly absorbed by the oil-absorbing sheet 10. More specifically, the oil-absorbing sheet 10 has an image region (11 to 13) and the other region (an oil-absorbing non-image region where the image-forming oil is not printed) and since the sebum is absorbed in both regions, the user can easily confirm the fact or degree of oil absorption by the change in the transparency or color of these regions. Furthermore, the oil-absorbing sheet 10 of the present invention has image regions 11 to 13 where a specific oil agent is further filled in voids, so that a visual effect based on the change in the transparency or color of these image regions can be observed. That is, the change in transparency or color differs between the image region and the non-image region or the picture pattern in the image region changes, for example, the picture pattern itself disappears (more specifically, the picture pattern is integrated with the color of the non-image region and essentially disappears), so that the user can enjoy the process of change of the picture pattern. Furthermore, with the image region a small amount of sebum being absorbed in the oil-absorbing sheet in the image region causes a change in transparency or color of the image region therefore a user having a small amount of sebum can clearly recognize the oil absorption effect.

The oil-absorbing cosmetic sheet of the present invention is first characterized in that a plastic material is used as the substrate not a paper substrate formed of vegetable fibers or the like as in conventional oil-absorbing paper. The plastic material is formed into a porous stretched plastic film. The porous stretched plastic film may be produced according to various techniques by using a plastic material as the starting material but is preferably produced by adding a filler to a crystalline thermoplastic resin having high transparency, and stretching the film to impart fine voids.

In the resulting porous stretched plastic film, the ratio of a space occupying by the voids of the sheet is large as compared with conventional oil-absorbing paper. This ensures excellent oil absorption capacity for sebum on the skin surface and a remarkably large oil absorption capacity per unit area. This plastic film also generally has a large number of fine voids of uniform structure and therefore, appears opaque due to diffusion of light before wiping sebum on the skin surface, but after oil absorption, the sebum fills the voids, which prevents or reduces the diffusion of light. This effect in combination with the transparency of the original film allows oil absorption to be clearly seen.

In producing the porous stretched plastic film of the present invention, preferred examples of the crystalline thermoplastic resin with high transparency include, but are not limited to, polyolefins such as high-density polyethylene, polypropylene, polybutylene, poly-4-methylpentene and ethylene-propylene block copolymer. Preferably, a high melt strength modified polyolefin.

To impart excellent softness (touch) or shapability to the oil-absorbing sheet, a polyethylene resin is particularly preferred as a raw material. The polyethylene resin used is not particularly limited but suitable examples of the polyethylene resin include, but are not limited to, high-density polyethylene, low-density polyethylene, linear low-density polyethylene, ultrahigh molecular weight polyethylene, polypropylene, polybutylene, poly-4-methylpentene, polyester and an ethylene-propylene block copolymer. These polyethylene resins may be used alone, or two or more thereof may be used as a copolymer or a mixture. In particular, a combination of high-density polyethylene and ultrahigh molecular weight polyethylene is optimally used as a raw material because of high melt strength during film formation. The resin preferably comprises the above-described polyethylene resin but, resins of different types may be used in combination with such polyethylene resins.

The filler used in combination with the thermoplastic resin for imparting fine voids is also not particularly limited, and an organic filler and/or an inorganic filler may be used. Suitable examples of organic fillers include, but are not limited to, mineral oils, liquid paraffins, glycerin, petroleum jelly, polyethylene oxide, polypropylene oxide, polytetramethylene oxide and soft carbowax. Among these organic fillers, mineral oil is preferred in view of the end transparency of the resulting film after oil absorption. Suitable examples of the inorganic filler include, but are not limited to, calcium carbonate, barium sulfate, titanium oxide and talc. These fillers may be used alone, or two or more thereof may be used as a mixture.

In production of the porous film, the filler can be added in various amounts to the resin. The amount of the filler added is preferably from about 20 to 90 wt %, more preferably from 40 to 80 wt %, and most preferably from about 25 to 40 wt %, based on the total weight of raw materials. If the amount of the filler added is less than 20 wt % based on the raw materials, the film obtained after stretching decreases in the void content and the oil absorption amount in turn decreases, whereas if it exceeds 90 wt %, film formation difficult and only fragile films can be obtained.

In producing the porous stretched plastic film, if desired, arbitrary additives may be used in combination with the above-described main raw materials. For example, an organic nucleating agent or an inorganic nucleating agent may be added for the purpose of facilitating the crystallization of the resin. Suitable examples of the organic nucleating agent include an organic acid such as carboxylic acid, sulfonic acid and phosphonic acid, and an organic alcohol. Suitable examples of the inorganic nucleating agent include titanium oxide, calcium carbonate and talc. Other examples of suitable additives include pigments, e.g., inorganic pigment or organic pigment, an aging resistor, an antioxidant, an antistatic agent and a perfume. Such additives are usually used in an amount of about 0.5 to 10 wt % based on the total weight of raw materials.

Of these additives, particularly useful are pigments, because by the addition of a pigment, the lightness of the oil-absorbing sheet of the present invention can be set to 50.0 to 90.0 in terms of the L* value of the CIE L*a*b* color system and in turn, the oil absorption effect can be very easily and exactly confirmed. If desired, a pigment and a dye may be used in combination.

In the oil-absorbing sheet, the oil absorption effect is usually confirmed by comparing the difference of transparency or color between the sheet (porous stretched film) before oil absorption and the sheet after oil absorption, particularly by recognizing the difference of lightness with a naked eye. Here, it is convenient to express the lightness of the oil-absorbing sheet by the L* value of the CIE L*a*b* color system. If the ΔL* value of the oil-absorbing sheet, (that is, the difference obtained by subtracting the L* value of the sheet after oil absorption from the L* value of the sheet before oil absorption), is larger, the oil absorption effect can be more clearly and easily confirmed. The pigment added is not limited in its kind or added amount, but suitable examples of the pigment include an inorganic pigment such as iron oxide, carbon black and ultramarine, and an organic pigment such as phthalocyanine blue, quinacridone red, azo-based red, monoazo yellow, phthalocyanine green and molybdate orange. These pigments may be used alone, or two or more thereof may be used as a mixture. The added amount of the pigment is preferably an amount of giving an L* value of about 50.0 to 90.0. Such an added amount of the pigment cannot be unconditionally specified because the L* value fluctuates depending on the color tone or the like of the pigment, but in general, the pigment is preferably added in an amount of 0.5 to 10 wt % based on the total weight of raw materials, similarly to other additives.

In the oil-absorbing sheet of the present invention, the oil absorption effect can also be confirmed from another aspect. That is, in a predetermined image formation region of the plastic film, an image-forming oil agent, in an amount imparting a picture pattern to the plastic film, is printed to at least partially fill voids of the plastic film and thereby form an image region. Based on the change in transparency or color, peculiar to this image region, and based on the difference in the change of transparency or color between the image region and the non-image region (where the image-forming oil agent is not printed,) the specific oil absorption effect of the present invention can also be confirmed.

In order to obtain a porous stretched plastic film containing a predetermined amount of a filler, the above-described raw materials and additives are melt-mixed and film-formed. The melt-mixing step and the subsequent film-forming steps can be performed according to conventional methods. Suitable examples of the melt-mixing include mixing by a single-screw or twin-screw extruder, and kneading by a kneader. Suitable film-firming methods include blown film forming or casting a film. For example, with blown films the raw materials are melt-mixed and blown from a circular die, whereby a cylindrical film can be formed. With cast films, the raw materials are melt-mixed and then extruded onto a chill roll (cooling roll) from a die, whereby a film is formed.

The plastic film after film formation is then stretched to impart fine voids to the plastic film. The stretching step can be performed according to conventional methods, similar to the film-forming step. Suitable examples of stretching methods include uniaxial stretching and biaxial stretching. The stretching draw ratio is, in terms of the surface draw ratio, preferably from about 1.5 to 36, more preferably from about 2 to 25, and most preferably from about 3 to 10. If the draw ratio is less than 1.5, the void content and volume is not large enough to allow for oil absorption, whereas if it exceeds 36 times, stretching becomes difficult and the film tends to be hard.

Although the stretching step is not limited to the following conditions, the stretching is preferably performed such that the thickness of the film after stretching is from about 5 to 200 μm. If the film thickness is less than 5 μm, the interstitial volume, and in turn the oil absorption amount, decreases and the film loses rigidity and adheres to the face, fingertip or the like, whereas if the thickness exceeds 200 μm, the oil absorption capacity is excessively large and this makes it difficult to confirm the oil absorption effect.

In the present invention, the filler may remain in the voids of the porous stretched plastic film, or with organic fillers a part of the filler may be selectively removed from the voids. The filler may be removed, for example, after film formation and before stretching, or during or after stretching. Also, the filler may be removed in a separate step independent of film formation. However, for the purpose of simplifying the process, in the case of removing filler, it is advantageous to remove the filler after the stretching step.

The removal of the filler may be performed by various methods, but a useful method in general is to wash the organic filler-containing porous plastic film with a solvent and remove the organic filler by dissolution or extraction. The solvent used here may be arbitrarily varied in relation to the filler to be removed, but examples of the solvent which is usually used include trifluoroethane, heptane, toluene, methyl ethyl ketone, isopropyl alcohol, ethyl alcohol, water, an acid and an alkali. These solvents may be used alone, or two or more solvents may be mixed and used. Examples of methods for applying the solvent to the plastic film include impregnation, a spraying, a showering and coating.

In the obtained porous stretched plastic film, the void content of the film can be defined by interstitial volume. That is, the interstitial volume per unit area (1 cm²) of the porous stretched plastic film is about 0.0001 cm³/cm² or more as calculated according to the following formula:

Interstitial volume per unit area=[film thickness (cm)×1 (cm)×1 (cm)×void content(%)]+100

In other words, it is preferred to selectively remove a part of the filler in the production process of the porous stretched plastic film so that the above-described interstitial volume can be obtained. Of course, if desired, such an interstitial volume can also be obtained while leaving the filler.

In the porous stretched plastic film, the interstitial volume per unit area (1 cm²) of the film is at least about 0.0001 cm³/cm², preferably from about 0.0001 to 0.005 cm³/cm², more preferably from about 0.0002 to 0.001 cm³/cm². If the interstitial volume of the porous film is less than 0.0001 cm³/cm², the oil absorption amount decreases, whereas if it exceeds 0.005 cm³/cm², the oil absorption amount becomes excessively large and this makes it difficult to confirm the oil absorption effect.

As for the void content of the porous stretched plastic film, the production conditions are preferably adjusted to give a void content of about 5 to 50%. If the void content is less than 5% after film stretching, the oil absorption capacity of the finally obtained oil-absorbing sheet is too low, whereas if the void content exceeds 50%, the maximum oil absorption capacity becomes too large making it difficult to confirm the oil absorption effect. However, in the implementation of the present invention, since the oil agent will at least partially filled in the voids in the image formation region, if desired, the void content may exceed 50%. The void content is generally from about 5 to 75%.

The porous stretched plastic film is not particularly limited for the void pore diameter. Generally speaking, the pore diameters of the voids is preferably from about 0.2 to 5 μm. If the average pore diameters are less than 0.2 μm, sebum in an amount necessary for transparentization may not be satisfactorily absorbed, whereas even if it exceeds 5 μm, significant improvement of the oil absorption effect is not obtained.

The oil-absorbing cosmetic sheet of the present invention usually comprises only the above-described porous stretched plastic film, but if desired, the plastic film may be subjected to an additional treatment. For example, when hydrophilicity is imparted to the surface of the plastic film, this provides an effect that sweat on the face or sebum dissolved therein can be easily absorbed in the oil-absorbing sheet. The surface treatment may be performed, for example, such that a hydrophilic liquid absorbing substance is at least partially distributed on at least one surface of the porous stretched plastic film.

In the oil-absorbing cosmetic sheet of the present invention, after the formation of the porous stretched plastic film, an image-forming oil agent needs to be applied to the plastic film to form an image region having a picture pattern (image pattern). That is, an image-forming oil agent in an amount of at least partially filling the voids already present in the plastic film and imparting a picture pattern to the plastic film is applied to the predetermined image formation region of the plastic film to form an image region. The picture pattern is not particularly limited and includes illustration, pattern, letter, numeric character, photograph and an arbitrary combination thereof. Also, the pattern may be colored with a single color or with two or more colors. In the case of coloring the pattern, an appropriate color is preferably decided by taking into account the color in the non-image region.

The image-forming oil agent may be filled in almost all voids in the image region or may be selectively filled in a part of the voids, according to the desired picture pattern. The oil agent is filled in voids and the filled state is stably maintained. Therefore, the oil-absorbing cosmetic sheet is not tacky during production, storage, or use. The sheets also do not block to each other.

The viscosity of the image-forming oil agent is preferably at least 10 cp when measured at 20° C. When an oil agent having a viscosity of less than 10 cp is applied to the plastic film, movement of the oil agent in the obtained oil-absorbing sheet occurred within a few days after formation and the picture pattern disappears. The upper limit for viscosity is not specified because various oil agents can be used if a suitable solvent is used in combination with the oil agent.

In the oil-absorbing sheet of the present invention, after the oil agent is applied to the plastic film, the ΔL* value between the image region to which the image-forming oil agent is applied and the non-image region to which the image-forming oil agent is not applied is preferably at least about 2.3 for maximally differentiation of the picture pattern. The ΔL* value is more preferably at least about 3.2. The ΔL* value becomes smaller as the amount of the oil agent within the voids increases. With this, it is possible to create a plurality of regions (image regions and non-image regions) differing in ΔL* values and differing in transparency or color change by the absorption of sebum. As a result, the sheets can be designed so that any user can clearly confirm the oil absorption effect, even a user having a relatively small amount of sebum.

In preparing the oil-absorbing cosmetic sheet of the present invention, various materials can be used as the image-forming oil agent. In view of handleability and application, liquid materials are generally preferred. Also, considering that the oil-absorbing sheet directly contacts the skin of a user, the material used is preferably not irritating to the skin. Furthermore, the material is preferably colorless and odorless and preferably has no moisture absorptivity.

In the practice of the present invention, many kinds of oils and fatty acid derivatives thereof can be used as the image-forming oil agent. A vegetable oil, a mineral oil or a blend thereof is preferred as the oil agent. Examples of vegetable oils include, but are not limited to, apricot kernel oil, avocado oil, baobab oil, black currant oil, Calendula officinalis oil, cannabis sativa oil, canola oil, chaulmoogra oil, coconut oil, corn oil, cottonseed oil, grape seed oil, hazel nut oil, hybrid sunflower oil, hydrogenated coconut oil, hydrogenated cottonseed oil, hydrogenated palm kernel oil, jojoba oil, kiwi seed oil, kukui nut oil, macadamia nut oil, mango seed oil, meadowfoam seed oil, Mexican poppy oil, olive oil, palm kernel oil, partially hydrogenated soybean oil, peach kernel oil, peanut oil, pecan oil, pistachio nut oil, pumpkin seed oil, quinoa oil, rapeseed oil, rice bran oil, safflower oil, sasanqua oil, sea buckthorn oil, sesame oil, shea butter fruit oil, sisymbrium irio oil, soybean oil, sunflower seed oil, walnut oil and wheat germ oil. Other examples of the oil which can be used as the oil agent include, but are not limited to, an oil having vitamin-like properties, such as cod liver oil, shark liver oil, menhaden oil, mink oil and palm oil; an oil having skin protective properties, such as carrot oil, echium plantagineum seed oil and fomistopsis officinalis oil; an oil having skin-conditioning properties, such as borage seed oil, cohune oil, lesquerella fendleri oil, passionflower oil, passionfruit seed oil and sweet almond oil; an oil having neutralizer properties, such as pine oil; an oil having moisturizer properties, such as aloe vera oil, babassu oil, brazil nut oil, camellia japonica oil, chia oil, ganoderma lucidum oil, hydrogenated castor oil, sweet cherry pit oil and tea oil; an oil having emulsifier properties, such as neatsfoot oil, neem seed oil, PEG-5 hydrogenated castor oil, PEG-40 hydrogenated castor oil, PEG-20 hydrogenated castor oil isostearate, PEG-40 hydrogenated castor oil isostearate, PEG-40 hydrogenated castor oil laurate, PEG-50 hydrogenated castor oil laurate, PEG-5 hydrogenated castor oil triisostearate, PEG-20 hydrogenated castor oil tristearate, PEG-40 hydrogenated castor oil tristearate, PEG-50 hydrogenated castor oil tristearate, PEG-40 jojoba oil, PEG-7 olive oil, PPG-3 hydrogenated castor oil, PPG-12-PEG-65 lanolin oil, hydrogenated mink oil, hydrogenated olive oil, lanolin oil, maleated soybean oil, musk rose oil, cashew nut oil, castor oil, dog rose hips oil, emu oil, evening primrose oil and gold-of-pleasure oil; an oil having dispersant properties, such as PEG-5 castor oil, PEG-9 castor oil, PEG-15 castor oil, PEG-25 castor oil, PEG-36 castor oil and PEG-18 castor oil dioleate; an oil having colorant properties, such as peppermint oil, spearmint oil and zedoary oil; an oil having buffer properties, such as chamomile oil and eucalyptus oil; a botanical oil such as balm mint oil; an oil having anti-microbial properties, such as tea tree oil; an oil having antioxidant properties, such as tocotrienols oil; an oil having fragrant properties, such as tangerine oil and lemongrass oil; a fatty acid derivative of oil, such as oleic acid, linoleic acid and lauric acid; and a substituted fatty acid derivative of oil, such as oleamide, propyl oleate and oleyl alcohol. These oil agents do not have volatility so high as to evaporate from the sheet between the production of the oil-absorbing sheet and use by a user.

Furthermore, a mineral oil or a synthetic oil can be used as the image-forming oil agent. Examples of the oil agent belonging to this category include, but are not limited to, a mineral oil, a petroleum, a linear or branched hydrocarbon and a derivative thereof.

Such an oil agent is applied to the porous stretched plastic film in various amounts according to the factors such as void content of the plastic film, desired picture pattern and oil absorption indicating effect, whereby an oil-absorbing sheet with a picture pattern can be produced. The amount of the oil agent applied is an amount sufficiently large to at least partially fill voids of the plastic film and impart a desired picture pattern to the film.

The preferred amount of the oil agent applied varies, as described above, depending on various factors and cannot be unconditionally specified but is usually from about 0.1 to 1 mg/cm². In the case where the plastic film has a plurality of image regions associated with each other, the same or different image-forming oils may be applied to respective image regions in the same or different amounts.

The image-forming oil agent may be applied to the porous stretched plastic film by various techniques. For example, a coating method is advantageously used and it is particularly recommended to apply the coating agent by a printing method. Suitable examples of printing methods include flexographic printing and gravure printing. In practicing such a printing method, a solvent is sometimes used for dissolving the oil agent but from the aspect of safety, an ethanol, an isopropyl alcohol or the like is preferably used. In combination with the oil agent, for example, a stabilizer such as an antioxidant may be used, because many vegetable oils are prone to oxidation.

The oil-absorbing cosmetic sheet of the present invention can be provided in various forms and is generally provided, for example, in the form of a disposable package of oil-absorbing sheets. Individual oil-absorbing sheets in the package are arranged in a stacked manner. The “stacked” means that one surface of one oil-absorbing sheet covers the entire or substantial portion of one face of an adjacent oil-absorbing sheet in the package. Generally, one package contains at least 2 or more oil-absorbing sheets, preferably about 10 to 1,000 oil-absorbing sheets.

The oil-absorbing sheet may have any suitable size, but in most uses, the oil-absorbing sheet generally has a surface area of about 10 to 100 cm², preferably from about 20 to 50 cm². In this way, the oil-absorbing sheet has a size suitable for insertion in a package, and the package can be easily placed in the bag or pocket of a user. The material forming the disposable container is generally not important, and the container may be formed of, for example, a suitable paper, plastic or paper-film laminate. The shape of the disposable container is generally rectangular, but other suitable shapes such as oval or circular can also be used.

### EXAMPLES

The present invention is described below by referring to examples. In the following examples, unless otherwise indicated, the “parts” means “parts by weight”. Also, it should be understood that the present invention is not limited to the following examples.

### Production Example

### Production of Porous Stretched Plastic Film

The following raw materials each in the blended amount shown below were charged into a twin-screw extruder and melt-mixed.

| | |
|---|---|
| Polypropylene resin (available under the trade name | 63.3 parts |
| of “5D45” from Union Carbide) | |
| Mineral oil (available under the trade name of “White | 34.0 parts |
| Mineral Oil #31” from Amco Oil & Chemical Co.) | |
| Organic nucleating agent (available under the trade name | 2.75 parts |
| of “Blue P-526” from Hoechst Celanese) | |

Subsequently, the obtained melt mixture was cast on a cooling roll from an extrusion die to form a film, and this film was biaxially stretched (1.8 times in the longitudinal direction and 1.8 times in the width direction). The resulting blue porous stretched plastic film had the following characteristics.

| Thickness | 35 | μm |
|---|---|---|
| Void content | 25% | |
| Interstitial volume per unit area | 0.000875 | cm³/cm² |

This blue-colored porous stretched plastic film had very soft touch and was usable as an oil-absorbing sheet without being subjected to a further treatment.

### Selection of Image-Forming Oil Agent

The porous stretched plastic film produced as above was cut into a rectangular shape of 9 cm (length)×6 cm (width) to prepare a sample film.

Separately, as shown in Table 1 below, 9 kinds in total of oily liquids (Comparative Examples 1 to 5 and Examples 1 to 4) differing in the viscosity at 20° C., which are generally known as a cosmetic raw material, were prepared. Subsequently, for producing an oil-absorbing cosmetic sheet, each oily liquid was flexo-printed in a pattern of polka-dots with a diameter of 1 cm on one surface of the sample film. The printing conditions were standardized such that the ΔL* value of the sample film between before and after printing of the oily liquid became 4. Incidentally, the ΔL* value was measured by the following procedure.

### Measurement of ΔL* Value

The chromaticity (initial L* value) of the sample film before printing the oily liquid was measured by a calorimeter (“SZ-EΣ80”, trade name, manufactured by Nippon Denshoku Kogyo Co.) according to the reflection process. Thereafter, the chromaticity (L* value after oil filling) after printing the oily liquid was measured similarly by the reflection process. From these measured values, the ΔL* value was determined according to the following formula:

ΔL*Value=initial L*value−L*value after oil filling

In order to confirm the stability of the oily liquid printed on the sample film, the sample film was stored in an oven at 60° C. and the change in aging of the polka-dot pattern printed on the oil-absorbing sheet (presence or absence of disappearance) was evaluated with an eye.

Also, 20 monitor persons were collected and the color, odor, feeling on use and the like of the oil-absorbing sheet were evaluated by individual persons. Also, the oil-absorbing sheet was rated good when gave an impression on use that sebum was very successfully absorbed, rated acceptable when gave an impression that sebum was absorbed to a certain extent, and rated unacceptable when gave an impression that sebum was not so much absorbed. Finally, based on evaluation results of “Stability after Printing” and “Feeling on Use”, overall judgment on a three-stage scale of O (good), Δ (acceptable) and x (unacceptable) was made. The evaluation results and judgment results obtained are shown in Table 1 below.

TABLE 1

| | Name of | | | viscosity, cp | | Feeling on | |
|---|---|---|---|---|---|---|---|
| | Chemical | Name of Product | Distributor | @20° C. | Stability after Printing | Use, etc. | Judgment |
| Comparative | liquid paraffin 1 | MORESCO- | Matsumura | 4 | disappeared at 60° C. in 1 | good | x |
| Example 1 | | VIOLESS P-40 | Oil Research | | day | | |
| Comparative | isopropyl | IPP | Croda Japan | 5 | disappeared at 60° C. in 3 | good | x |
| Example 2 | myristate | | | | days | | |
| Example 1 | liquid paraffin 2 | MORESCO- | Matsumura | 10 | slightly faded at 60° C. in | good | Δ |
| | | VIOLESS P-55 | Oil Research | | 5 days but not | | |
| | | | | | disappeared for 30 days | | |
| | | | | | or more | | |
| Example 2 | caprylic capric | GTCC | Croda Japan | 17 | stable at 60° C. for 30 | good | ∘ |
| | acid triglyceride | | | | days or more | | |
| Example 3 | liquid isoparaffin | PARLEAM 6 | NOF | 25 | stable at 60° C. for 30 | good | ∘ |
| | | | | | days or more | | |
| Example 4 | olive oil | Olive Oil | Iwase Cosfa, | 60 | stable at 60° C. for 30 | good | ∘ |
| | | | etc. | | days or more | | |
| Comparative | polyether | UNILUB MB-7 | NOF | 72 | stable at 60° C. for 30 | smell | x |
| Example 3 | | | | | days or more | | |
| Comparative | polypropylene | Polypropylene | Sanyo | 143 | stable at 60° C. for 30 | sticking on | x |
| Example 4 | glycol | Glycol 950 | Chemical | | days or more | surface due to | |
| | | | | | | moisture | |
| | | | | | | absorption | |
| Comparative | nonionic | NONION OP- | NOF | 250 | stable at 60° C. for 30 | smell | x |
| Example 5 | surfactant | 85R | | | days or more | | |

It is seen from the results in Table 1 that the oily liquid having a viscosity of 5 cp or less causes disappearance of the polka-dot pattern in a few days after heating and is not suitable for use as the image-forming oil agent. On the other hand, the oily liquid having a viscosity of 10 cp or more can stably maintain the polka-dot pattern even under severe heating conditions and is suitable as the image-forming oil agent. Also, some oily liquids smell and another oily liquid has moisture absorptivity, which are all unsuited for the implementation of the present invention.

### Printing Test 1

According to the procedure described in Production Example above, 6 kinds in total of porous stretched plastic films differing in the color as shown in Table 2 were produced and cut into a rectangular shape of 9 cm (length)×6 cm (width) to prepare sample films.

Subsequently, caprylic/capric triglyceride (GTCC, produced by Croda Japan), which is the oily liquid of Example 2 having a viscosity of 17 cp at 20° C., was prepared and printed on the sample film by a 175 line gravure printing press. The gravure printing press was equipped with a gravure roll having line sections of giving gradations of 30, 50, 70, 90 and 100%.

The chromaticity (initial L* value) of the sample film before printing the oily liquid and the chromaticity (L* value after oil filling) after printing the oily liquid were measured by a calorimeter (“SZ-EΣ80”, trade name, manufactured by Nippon Denshoku Kogyo Co.) according to the reflection process and from these two measured values, the ΔL* value was determined.

Also, by placing the sample film to stand against a black plate, the sharpness of each line image was judged with an eye. The Δ* values and judgment results obtained are shown in Table 2 below.

TABLE 2

| Film Color | Light Blue | | White | | Dark Blue | | Ultramarine | | Apricot | | Green | |
|---|---|---|---|---|---|---|---|---|---|---|---|---|
| Gradation | ΔL* | Visual | ΔL* | Visual | ΔL* | Visual | ΔL* | Visual | ΔL* | Visual | ΔL* | Visual |
| 30 | 2.58 | Δ | 1.02 | x | 2.60 | Δ | 2.38 | Δ | 2.66 | Δ | 1.40 | x |
| 50 | 4.49 | ∘ | 2.01 | x | 4.12 | ∘ | 4.56 | ∘ | 3.20 | ∘ | 2.39 | Δ |
| 70 | 6.41 | ∘ | 0.94 | ∘ | 6.71 | ∘ | 6.51 | ∘ | 5.34 | ∘ | 3.87 | ∘ |
| 90 | 9.32 | ∘ | 4.90 | ∘ | 8.05 | ∘ | 9.80 | ∘ | 6.82 | ∘ | 5.10 | ∘ |
| 100 | 36.39 | ∘ | 28.25 | ∘ | 29.72 | ∘ | 36.23 | ∘ | 35.54 | ∘ | 35.71 | ∘ |

ΔL*: Difference of L* between non-printed portion and printed portion.
Criteria of judgment with eye:
∘: The pattern is clearly visible.
Δ: The pattern is visible.
x: The pattern is scarcely visible.

It is seen from the results in Table 2 that at the visual test, when the ΔL* value is 2.3 or more, the line image (pattern) is visible and when the ΔL* value is 3.2 or more, the pattern is more clearly visible.

### Printing Test 2

The test was performed according to the procedure described in Printing Test 1 above but in this example, 5 monitor persons A to E differing in the amount of sebum on the face were collected. Also, adjacently arranged Areas 1, 2 and 3 (each in 1 cm×1 cm) were provided on one sample film and caprylic/capric acid glyceride (GTCC, produced by Corda Japan), which is the oily liquid of Example 2 having a viscosity of 17 cp at 20° C., was printed by a gravure printing press. The gradation of the gravure roll was changed among the Areas and was 50% in Area 1, 70% in Area 2 and 90% in Area 3. Furthermore, from the chromaticity (initial L* value) of the sample film before printing the oily liquid and the chromaticity (L* value after oil filling) after printing the oily liquid, the ΔL* value was determined and found to be 4.1 in Area 1, 6.7 in Area 2 and 8.1 in Area 3.

Subsequently, by using the sample film as an oil-absorbing cosmetic sheet, an actual application test was performed by 5 monitor persons. Each sample film was observed with an eye to check whether or not Areas 1, 2 and 3 could be transparentized by absorbing sebum. The judgement results obtained are shown in Table 3 below.

TABLE 3

| | ΔL* | Monitor Person A | Monitor Person B | Monitor Person C | Monitor Person D | Monitor Person E |
|---|---|---|---|---|---|---|
| Area 1 | 2.5 | not transparentized | not transparentized | transparentized | not transparentized | not transparentized |
| Area 2 | 5 | not transparentized | transparentized | transparentized | not transparentized | not transparentized |
| Area 3 | 7.5 | transparentized | transparentized | transparentized | transparentized | transparentized |

| | | | | | |
|---|---|---|---|---|---|
| Judgment | little sebum | normal sebum | much sebum | little sebum | little sebum |

It is seen from the results in Table 3 that transparentization is generated or not generated in Areas 1 to 3 even in the case of one monitor person and since the amount of sebum differs among the monitor persons, the transparentization also fluctuates among monitor persons. In other words, when the oil-absorbing sheet of the present invention is used, whether the sebum amount is large or small can be judged at a glance for respective monitor persons and in turn, for all users.

1-7. (canceled)

8. An oil-absorbing cosmetic sheet comprising a porous stretched film formed of a plastic material having a predetermined image formed from an image-forming oil agent, wherein the image-forming oil agent is applied in an amount to impart a visible picture pattern to said plastic film by at least partially filling voids of the plastic film.

9. The oil-absorbing cosmetic sheet as claimed in claim 8, wherein the viscosity of said image-forming oil agent is at least 10 cp at 20° C.

10. The oil-absorbing cosmetic sheet as claimed in claim 8, wherein in said plastic film, the ΔL* value between said predetermined image and a non-image region to which said image-forming oil agent is not applied is at least 2.3.

11. The oil-absorbing cosmetic sheet as claimed in claim 10, wherein a plurality of predetermined images are formed, and wherein the same or different image-forming oil agents in the same or different amounts are applied to form respective predetermined images.

12. The oil-absorbing cosmetic sheet as claimed in claim 10, wherein in said plastic film, the interstitial volume per unit area (1 cm²) of the film is from 0.0001 to 0.005 cm³/cm² as calculated by the following formula:
interstitial volume per unit area=[film thickness (cm)×1 (cm)×1 (cm)×void content (%)]+100.

13. The oil-absorbing cosmetic sheet as claimed in claim 10, wherein the void content of said plastic film is from 5 to 50%.

14. The oil-absorbing cosmetic sheet as claimed in claim 10, wherein the thickness of said plastic film is from 5 to 200 μm.

15. The oil-absorbing cosmetic sleet as claimed in claim 10, wherein the viscosity of said image-forming oil agent is at least 10 cp at 20° C.

16. The oil-absorbing cosmetic sheet as claimed in claim 8, wherein a plurality of predetermined images are formed, and wherein the same or different image-forming oil agents in the same or different amounts are applied to form respective predetermined images.

17. The oil-absorbing cosmetic sheet as claimed in claim 16, wherein the viscosity of each image-forming oil agent is at least 10 cp at 20° C.

18. The oil-absorbing cosmetic sheet as claimed in claim 16, wherein in said plastic film, the interstitial volume per unit area (1 cm²) of the film is from 0.0001 to 0.005 cm³/cm² as calculated by the following forma:
interstitial volume per unit area=[film thickness (cm)×1 (cm)×1 (cm)×void content (%)]+100.

19. The oil-absorbing cosmetic sheet as claimed in claim 16, wherein the void content of said plastic film is from 5 to 50%.

20. The oil-absorbing cosmetic sheet as claimed in claim 16, wherein the thickness of said plastic film is from 5 to 200 μm.

21. The oil-absorbing cosmetic sheet as claimed in claim 16, wherein at least one of different image-forming oil agents or different amounts of image-forming oil agent are applied to form respective predetermined images.

22. The oil-absorbing cosmetic sheet as claimed in claim 8, wherein in said plastic film, the interstitial volume per unit area (1 cm²) of the film is from 0.0001 to 0.005 cm³/cm² as calculated by the following formula:
interstitial volume per unit area=[film thickness (cm)×1 (cm)×1 (cm)×void content (%)]+100.

23. The oil-absorbing cosmetic sheet as claimed in claim 8, wherein the void content of said plastic film is from 5 to 50%.

24. The oil-absorbing cosmetic sheet as claimed in claim 8, wherein the thickness of said plastic film is from 5 to 200 μm.
